(19) United States
(12) Patent Application Publication
(10) Pub. No.: US 2023/0016321 A1
(43) Pub. Date: Jan. 19, 2023

(54) USES AND APPLICATIONS IN CANCER AND OTHER PROLIFERATIVE CONDI-TIONS OF ONCOPROTEIN CSP80 AND AGENTS INTERACTING WITH ONCOPROTEIN CSP80

(71) Applicants: Ahmed Fadiel-Metwaly, Smyrna, TN (US); Frederick Naftolin, Woodbridge, CT (US)

(72) Inventors: Ahmed Fadiel-Metwaly, Smyrna, TN (US); Frederick Naftolin, Woodbridge, CT (US)

(21) Appl. No.: 17/864,713

(22) Filed: Jul. 14, 2022

Related U.S. Application Data
(60) Provisional application No. 63/221,742, filed on Jul. 14, 2021.

(51) Int. Cl. G01N 33/574

(52) U.S. Cl. CPC G01N 33/57488

(57) ABSTRACT

The methods of the present invention provide a new diagnostic marker for proliferative conditions and cancers, such as ovarian cancer, endometrial cancer and others. A truncation of MAP1a, CSP80 is an oncoprotein identified as being present in bodily fluids, or contents thereof, of a subject when afflicted with a proliferative condition. The present invention disclosed diagnosis and monitoring of a proliferative condition by detection of CSP80. Additionally, the present invention discloses a targeted treatment of proliferative conditions utilizing CSP80 binding agents, via a variety of delivery vehicles such as nanoparticles.

### REFERENCE TO RELATED APPLICATION

This application claims benefit of the filing date of U.S. Provisional Patent Application Ser. No. 63/221,742, entitled, “USES AND APPLICATIONS IN CANCER AND OTHER PROLIFERATIVE CONDITIONS OF AGENTS INTERACTING WITH THE UNIQUE MICROTUBULE ASSEMBLY PROTEIN CSP80 OR ABNORMAL MEMBERS OF THE ERM FAMILY OF PROTEINS,” and filed on Jul. 14, 2021. The teachings of the referenced application are herein incorporated by reference.

### FIELD OF THE INVENTION

The present invention discloses the use of agents that interact with the oncoprotein CSP80 and detect, affect and deliver treatment of cells that express this protein. For descriptive purposes, examples based on ovarian and endometrial cancer are depicted. Still, these are only examples rather than limits of the use of these agents on developing or actual human and animal cancers, metastatic cells, and proliferative disease cells. All pre-malignant, malignant and proliferative disease cells, their contents and fragments are targeted in this disclosure.

### BACKGROUND OF THE INVENTION

### The Problem of Cancer and Proliferative Diseases

Cancer is the second leading cause of death in the United States. Half of all men and one-third of all women in the U.S. will develop cancer. Today, millions of people are living with cancer or have had cancer. The sooner a cancer is found and treatment begins, the better the chances for survival. However, the pre-malignant and frankly malignant cells may escape early diagnosis, grow and metastasize, at which point the treatment becomes more difficult by orders of magnitude. Although diagnosis and treatment have improved, there is an urgent need for early diagnosis, improved clinical staging and safe, efficacious treatment. The same applies to proliferative diseases such as blood cell cancers, “benign” brain tumors that kill by displacing normal cells and such diseases as fibromatosis and psoriasis. To illustrate, ovarian cancer is preventable cancer in the ovaries' cells. The most common type of cancer arising from the ovary is ovarian epithelial cancer (OVCA) which may arise as a proliferative stage that leads to localized, “in situ” cancer and then invasive ovarian cancer or be the result of droplet seeding from fallopian tube cancer. A second, less common intraperitoneal cancer (idiopathic peritoneal cancer) apparently arises from the same peritoneum as covers the ovary and has a clinical course indistinguishable from OVCA.

Even in this era of increased interest in the health needs of women, OVCA stands out as a deadly disease in particular need of attention. Because of the lack of early symptoms, ⅔ of OVCA cases are metastatic at the time of detection and will not be cured. According to the American Cancer Society, ovarian cancer accounts for only 4% of all cancers among women but ranks fifth as a cause of their deaths from cancer. Over 14,000 women die each year from OVCA/PPC alone (2.3% of all cancer deaths), making it the leading cause of death from gynecologic malignancy. The reason for this late diagnosis the absence of suitable diagnostic tests. OVCA malignant cells along adjacent peritoneum and can progress extensively before they become symptomatic locally or their metastases cause symptoms. Pelvic examination or imaging is not successful in finding primary OVCA. Present markers, such as CA125, are non-specific and have false positives. The result of these failures is that OVCA is usually detected at an advanced stage and the outcome is almost uniformly fatal. No dependable method of discovery of OVCA is available and no curative drug treatment exists for metastatic OVCA. Despite the availability of new therapies, the mortality rate attributed to OVCA has not changed significantly in the last 50 years. One result of this situation is the increased performance of prophylactic ovariectomy. This ablation of the ovary, and often the uterus and Fallopian tubes, renders women infertile and often results in early death from premature menopause.

Endometrial adenocarcinoma (ENDOCA) is the most common gynecological cancer in women. It arises from the endometrial glandular lining and, unlike OVCA, it may metastasize by invading lymphatics and blood vessels to metastasize widely, as well as invading locally. ENDOCA begins as a proliferative condition, endometrial hyperplasia that undergoes malignant degeneration. The treatment of persistent hyperplasia often requires hysterectomy, resulting in infertility. Although ENDOCA cells are of a similar level of aggressivity as OVCA, ENDOCA is not as lethal because it causes uterine bleeding or an abnormal ultrasound examination that sets off a diagnostic workup that exposes the ENDOCA and results in treatment before metastases occur. However, there is no simple or painless or certain method of performing this sampling. Usually, an endometrial biopsy or curettage is required, and thus repeated/screening testing is not feasible in large populations. Even then, all forms of endometrial biopsy have a low but definite rate of false negatives.

Cervical cancer (CXCA) arises from the epithelium of the uterine cervix, generally at the juncture of the squamous and columnar glandular epithelium. CXCA is almost always associated with human papillomavirus infection and passes through a pre-invasive, proliferative stage. Early diagnosis requires sampling of the squamocolumnar junction. Although Pap smears have reduced the death rate of CXCA it has remained a major cause of women's deaths.

Thus, OVCA, ENDOCA and CXCA are examples of preventable cancers which pass through proliferative pre-malignant stages before they achieve symptoms to trigger detection by present methods of diagnosis. However, by then it is often too late for OVCA treatment and usually results in hysterectomy and infertility in the case of ENDOCA and CXCA. There is a need to develop new cancer diagnosis and treatment methods, particularly those for diagnosing and/or inhibiting cancer before the invasion and/or metastasis. Additionally, even after then, metastatic cell diagnosis, localization and treatment are lacking.

Treatments of OVCA, ENDOCA and CXCA are governed by the ability to properly define the extent and location of metastases. Currently, there are no tumor-specific agents to allow either external (x-ray/MRI/other imaging) identification of their proliferating malignant cells or to assist in the clinical staging at surgery or to assess the completeness of removal techniques (surgery/focused ultrasound, etc.). The planning and execution of treatment is hindered by the lack of specific agents that can be used to sample biological fluids or be incorporated in imaging techniques to assess the effect of treatment or the possibility of recurrence. Further, the lack of specific agents targeting molecules specific to cancer and other proliferative lesions has hindered the development of agents that safely and selectively mark and treat these lesions.

Evidence of Cancer Components in Biological Fluids

Until recently there has been a lack of evidence for components of cancer cells containing cancer-specific molecules being released from the cells themselves. This further hindered the above testing and methods for delivering of cancer-specific molecules for the above purposes. But it has now been shown that living and dead cancer cells regularly shed and absorb portions of their cells (cell fragments, micro-vesicles, exosomes, DNA, other molecules, etc.) that are found in intercellular spaces and biological fluids. These structures carry cancer-specific molecules that could identify/quantitate the presence of cancer and other proliferative disease cells. As well, such cancer-specific agents could be used to carry cargos of cancer-specific marking or treatment agents that would accomplish the tasks described above.

Microtubule-Associated Proteins, Cancer and Other Proliferative Diseases

Microtubules (MT) are constructed from segments of tubulin that are constantly being added to one end and removed from the other end. This is facilitated by microtubule-associated proteins (MAP's). The MT are bound to the cytoskeletal network of actin fibers. In this manner the coordinated shortening of one end and elongation of the other end of the microtubule regulates cell motility, the separation of chromosomes during cell division, the furnishing of cellular components to regions of the cell and other vital functions and the development of cellular processes that are critical to intercellular movement such as metastasizing. The microtubules play a role in the aberrant shapes and metastatic behavior of cancer cells through their attachment to the actin cytoskeleton, which is bound to the cell membranes by ezrin-radixin-Merlin (ERM) proteins.

Microtubule-associated proteins (MAPs) are necessary for the assembly of microtubules and their interaction with the cytoskeletal elements′, actin and the intermediate filaments. There are several families of MAPs—MAP1, MAP2, MAP4 and tau. MAP1 is unique in that it is only found in the nervous system while other MAPs are tissue-dependent in their distribution throughout the body. The MAPs bind to tubulin and also secure the microtubules to the other cytoskeletal components. In this manner the MAPs are in the area of the cell membrane of microvilli and other projections that may break off and find their way to the bodily fluids, or contents thereof. These dynamics are especially prominent in cancer and proliferative lesions.

### SUMMARY OF THE INVENTION

The disclosed invention is partly based on the discovery of an oncoprotein named CSP80 in cancers such as OVCA, ENDOCA, CXCA and other cancers. CSP80 may be present in proliferative lesions including blood cancers, fibromatoses, psoriasis, meningiomas, but is not expressed by normal cells in humans and animals. Other proliferative conditions include, but are not limited to, rheumatoid arthritis, atherosclerosis, idiopathic pulmonary fibrosis, scleroderma, cirrhosis of the liver, neovascularization or any other condition having a proliferation of cells. CSP80 may be a truncated form of the MAP1a or arise de novo. Since it is not expressed in normal cells, specific CSP80 binding agents such as antibodies or small molecules may be used in humans and animals for discovery and monitoring of cancer and other proliferative conditions; staging and clinical management or cancer and other proliferative conditions; cancer-targeting treatment and prevention of normal cells undergoing malignant transformation, pre-invasive (in situ) cancers, invasive cancers and other proliferative conditions.

Regarding discovery and monitoring of cancer and other proliferative conditions—specific antibodies to oncoprotein CSP80 may be used to identify and measure these proteins in bodily fluids to screen for the presence of precancerous and cancer tissues, or in risk cases to confirm the presence of CSP80 in an individual person or animal. The same measurements can be used to monitor the effects of treatment of cancers or other proliferative diseases that express CSP80.

As the present invention relates to cancer staging and clinical management-binding agents tagged with imaging-dense, chemiluminescent, fluorescing or radioactive materials may be used to detect cancers or other proliferating disease cells and masses by imaging or direct observation methods.

The present invention also relates to cancer targeting treatment-nanoparticles bound to normal glycocalyx antigens, such as the extracellular domains of receptors, and be internalized by endocytosis, after which they may be released from the endocytosis vesicles and bind to CSP80-target proteins via outer nanoparticle or small molecule-bound monoclonal antibodies, or nanoparticles may enter cells indiscriminately and then bind to CSP80 via specific monoclonal antibodies. The therapeutic agent cargos of the nanoparticles include chemotherapeutic drugs, microRNA's, toxic small molecules and radioactive isotopes. These actions depend on the presence of the target CSP80 protein; therefore, they should not affect normal cells.

The present invention also relates to cancer prevention—oncoprotein CSP80 expressed in apparently normal cells or tissues or cancerous tissues or those of proliferating diseases may be used to predict or actually be undergoing cryptic malignant degeneration or proliferative changes and may serve as a target for treatment-laden CSP80-binding molecules and biomaterials such as nanoparticles and small molecules.

Methods of the present invention can be used to detect and treat a variety of proliferative conditions, including solid cancers, blood cancers, sarcomas and (non-cancer) proliferative disorders.

The cancers and abnormally developing cells such as atypical hyperplasias, hamartomas, fibromas that can be detected or treated using the subject method include, but are not limited to ovarian cancer, endometrial cancer, breast cancer, glioblastoma, schwannoma, meningioma, malignant mesothelioma, neurofibromatosis, colon cancer, oral cancer, or cancer selected from the group consisting of: lung cancer, prostate cancer, pancreatic cancer, leukemia, liver cancer, stomach cancer, uterine cancer, testicular cancer, brain cancer, non-Hodgkin's lymphoma, Hodgkin's lymphoma, Ewing's sarcoma, osteosarcoma, neuroblastoma, rhabdomyosarcoma, melanoma, and metastatic tumors of the brain or other organs.

By way of example and not limitation, cancer being detected or treated by the present invention may be Atypical or malignant transforming, pre-invasive, non-invasive, invasive, and/or metastatic, hematologic, a solid tumor or solid tumor cancer of any organ or tissue, a carcinoma, a sarcoma, a myeloma, a leukemia, a lymphoma or any combinations thereof.

### BRIEF DESCRIPTION OF THE DRAWINGS

FIG. 1, shows the consensus amino acid sequence for CSP80 based on “walking PCR”.

FIG. 2, Western blot of MAP1ca in OVCA cell lines and OVCA clinical samples. Lane 1: SKOV3, lane 2: BIX3, lane 3: BIXLER, lane 4: DK2NMA, lane 5: ascitic OVCA cells, lane 6: papillary serous carcinoma, lane 7: ovarian epithelial carcinoma, lane 8: rat brain. An 80 kDa protein is present in lanes 2-8; the protein in lane 9 is 350 kDa.

FIG. 3, CSP80 expression in non-OVCA Cell Lines, CSP80 Expression in Breast & Endometrial Cancer Lanes: M: protein marker, 1: SKOV3 2: MCF7, 3: HEC.

FIG. 4, CSP80 in OVCA Specimens and Cell Lines Lanes: 1-3 OVCA tissue specimens, 4. Human brain, 5. DK2NMA—OVCA cells, 6. BIX3—OVCA cells, 7. SKOV3—OVCA cells, 8. BIXLER OVCA cells.

### DETAILED DESCRIPTION OF THE INVENTION

Currently, the oncogene/tumor suppressor gene mutation theory is a commonly accepted explanation of tumorigenesis. MAPs regulate cell growth and activities such as motility, invasion, size and rate of cell division. While these activities are what defines normal cells, abnormalities of MAPs could allow or cause abnormal cell activities or abnormalities of these activities. Microtubules actively participate in forming the mitotic spindle that plays the key role in the cell division, and abnormalities of the mitotic spindle may result in abnormal mitosis. Abnormal activities of MAPs could be an active process, or one in which truncated MAPs interfere with the normal function of MAPs the present invention/discovery is concordant with the mutant tumor suppressor gene theory.

Using multidisciplinary platforms (differential transcriptomics, proteomics and bioinformatics), a new molecule was discovered in cancer cells but not in normal cells or tissues. CSP80 is a novel MAP, i.e., it binds to tubulin and has domains similar to known MAP's, that is present in tissues undergoing increased cell proliferation and cell transformation/a malignant phenotype. CSP80 is not present in normal cells/tissues.

The cells of cancer, and other proliferative diseases, develop abnormal mitoses and strikingly unusual shapes and processes, related to malfunctioning of their microtubule-cytoskeletal dynamics. Microtubule-associated proteins (MAPs) participate in these interactions. As a strategy to discover molecules specific to ovarian epithelial carcinoma (OVCA), MAP expression in OVCA cells and tissues was studied. The instant invention discloses the presence of a low molecular weight MAP that shares a high level of DNA sequence similarity and immunoreactivity with MAP1a but is unique to OVCA and other cancers, but is not found in normal cells.

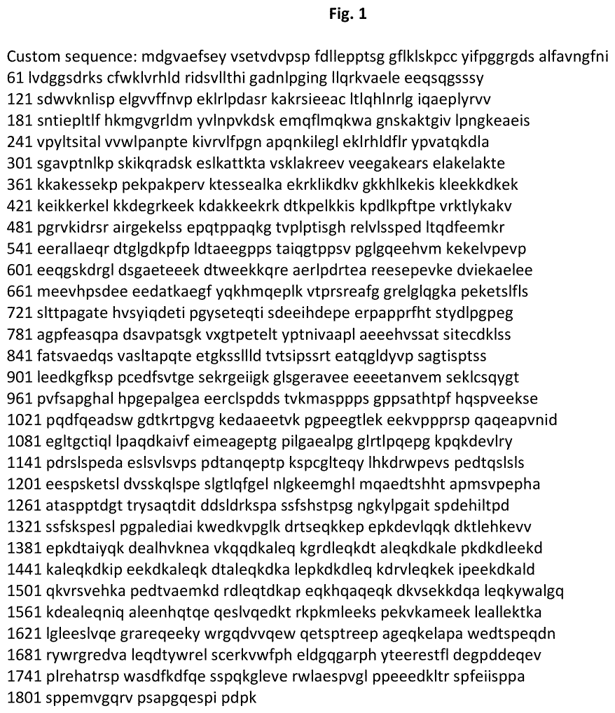

The present invention discloses the discovery of a MAP, termed CSP80, present in all cancers thus far studied, including OVCA and ENDOCA. CSP80 is absent in normal cells or organs. CSP80's amino acid composition has been derived by walking PCR plus computational DNA sequencing, FIG. 1. The findings regarding the presence of CSP80 and the absence of other MAPs are supported by immunohistochemical and Western analysis of cells, tissues, culture media and peritoneal fluid of OVCA, ENDOCA, and other cancer specimens.

In identifying CSP80, a Western Blot analysis was conducted on metastatic ovarian cancer specimens, normal human tissues and cancer specimens. All tissues were obtained under IRB-approved protocols. In this search for abnormal MAP-like proteins in cancers, normal human ovarian tissue, normal rat brain tissue and normal human brain tissues served as a MAP1a positive controls. A panel of anti-MAP monoclonal antibodies against the following proteins was used: MAP1a, MAP1b, MAP2a, MAP2b and anti-tau protein (Sigma, St. Louis), in the Western Blot analysis.

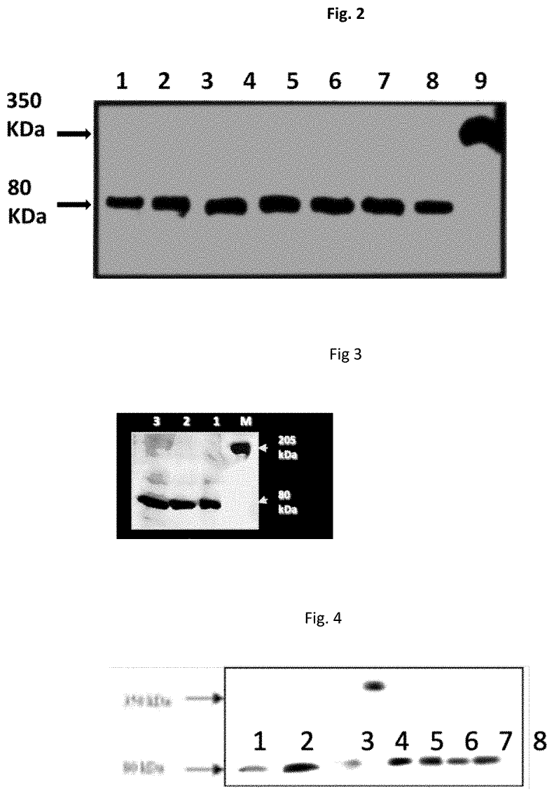

MAP1a was present as a single 350 kDa band in all tested brain extracts, FIG. 2. The anti-MAP1a immunostaining from OVCA extracts was restricted to a single 80 kDa band. No 350 kDa MAP1a immunoreactivity was found in the OVCA samples, despite its presence in concurrently-analyzed normal brain tissue-positive control extracts (band 9, FIG. 2). None of the other anti-MAPs tested showed more than a single band. The 80 kDa protein was termed CSP80. CSP80 appears to be a unique protein that contains epitopes found in MAP1a (not shown).

In multiple tests, no 80 kDa immunoreactivity was found in extracts of rat or human brain tissues or cultured rat or human glial cells, nor was it present in normal rat spleen, liver or lung (data not shown). On the contrary, CSP80 was the single band that was present in ovarian, breast and endometrial cancer, FIG. 3, FIG. 4.

The sequence of oncoprotein CSP80 was conducted by “walking PCR primer extension”, also known as Directed Sequencing, and is a sequencing method for assessing DNA fragments that are too long to be sequenced using the chain termination method. Sequential overlapping primers were derived from consecutive authentic sequences from wild-type MAP1A and tested against CSP80 extracts. This method confirmed the presence of the complete MAP1a RNA sequence in normal brain tissues, but not in other, non-neuronal samples. On the contrary, overlapping primers revealed a smaller-sized transcriptome in extracts from OVCA. Starting with the MAP1a 5′ terminal the sequence obtained from OVCA (CSP80) was homologous with the WT until 2603 bp but stopped at that point. The amino acid sequence of CSP80 is provided in FIG. 1.

CSP80 was cloned using different sets of sense and antisense primers sequencing analysis was performed in both directions. To minimize ambiguities, overlapping analysis revealed major homology between the sequence of the human brain MAP1a and the CSP80 sequence thus far obtained from SKOV3 ovarian cancer cells (not shown). But the cDNA sequencing analysis shows that although there is similarity between 80 kDa. CSP80 and the 350 kDa human brain MAP1a, insertion of four bp is present in the CSP80 cDNA that results in a premature stop codon and transcription of the mutated CSP80 protein. The origin of CSP80 is unknown at this time. It may be a result of a premature stop codon of MAP1a, it may be a post-translational modification of the same. Regardless of the origin of CSP80, the discovery of its presence provides significant opportunity to detect, monitor and treat proliferative diseases and cancers.

### Function of Oncoprotein CSP80

Additional efforts to determine the function of CSP80 led to further characterization, which revealed that the expression of CSP80 in cultured ovarian surface epithelial cells from normal-appearing ovaries was associated with transformation into malignant cells. CSP80 was not found in normal ovarian surface epithelial cells in tissue slices from normal ovary. This expression of CSP80 may be associated with the length of time in culture and the addition of estrogen and growth factor (insulin). Such an association would be consistent with the reported transformation of cultured ovarian surface epithelial cells from normal rat ovaries. These data were obtained in cultured cells; however, this may indicate an example of the above, that a mutant gene is expressed that interferes with normal MAP's acting as tumor suppressing proteins. This would result in transformation, or contribute to the transformation of normal cells and tissue to the pathology of a proliferative condition, tumor or cancer. Therefore, CSP80 may be a mutated tumor suppressor protein and is termed an oncoprotein.

These findings indicate that CSP80 and/or free-floating CSP80-containing structures present in bodily fluids such as ascitic fluid, endometrial secretion, blood or contents thereof, urine, serum, lymph fluid, endometrial washings from individual women or semen from men (CSP80 is expressed in prostate cancer) may reveal pre-malignant or malignant cells tissues in humans and animals, or a proliferative condition.

Description of Agents to Discover and Monitor CSP80 and. Diagnose a Proliferative Condition or Cancer

In one aspect, the invention provides a binding agent interacting with CSP80 in an individual with cancer or proliferative conditions. In another aspect of the invention, the binding agent interacting with, or binding to, CSP80 indicates the presence of a proliferative condition in a subject. In another aspect of the invention, the binding agent interacting with, or binding to, CSP80 detects the presence of a proliferative condition or a cancer and is used for diagnosis or monitoring of a proliferative condition or cancer.

Various CSP80 protein binding agents may be used in the instant invention. For example, the binding agent may be an antibody, or a functional fragment thereof. Antibodies (monoclonal antibodies, mAB) are amino acid-literate molecules that find and bind to “epitopes”, small groups of amino acids, that uniquely identify individual proteins. Monoclonal antibodies are specific in their interaction with a single, protein unique epitope. “Functional fragment” includes a fragment that binds the antigen, preferably binds the antigen and has at least one functional effect of the full antibody (such as inhibiting the function of the antigen or binding partner), especially when used in the context of the subject treatment method. However, functional fragments may only need to be able to bind their intended target molecule for the various diagnosis embodiments of the invention. The antibody may be a polyclonal antibody or a monoclonal antibody. The antibody may be a xenogeneic, an allogeneic, or a syngeneic antibody. The antibody can also be a modified antibody selected from the group consisting of a chimeric antibody, a humanized antibody, and a fully human antibody. The functional fragment of an antibody may be F(ab′)2, Fab, Fv, or scFv, one or more CDRs, etc.

Generating monoclonal antibodies, may be via in vitro or in vivo methods. Whereas protocols for generating monoclonal antibodies are well known in the art, a general description is provided by way of example and not limitation. Those skilled in the art may leverage varying protocols or organisms to the same end.

Generally, In vivo methods entail immunizing an animal, such as a mouse, rabbit or other animal, with a suitable antigen, CSP80, or an epitope thereof that may be desired for detection. The antigen is injected with an adjuvant, such as Freund's adjuvant. This leads to production of desired antibodies in the animal's body. The antigen may be injected into the animal multiple times. Typically, this immunization is done for a few weeks until the antibody concentration in the animal increases to the desired level.

After a desired number of weeks, the blood or contents thereof, or spleen, is obtained from the animal to assess the antibody titer, using techniques known in the art, such as ELISA or flow cytometry. The obtained monoclonal antibodies may be fused with an immortal cancer cell, such as myeloma, so that it may divide indefinitely, generating an antibody-producing hybridoma, which can be grown in vitro by tissue culture, or may be injected into a mouse peritoneal cavity to grow rapidly and can be harvested from ascites in the abdomen.

A general description of in vitro methods is provided by way of example and not limitation, for generating monoclonal antibodies, involves fusing activated antibody-producing B-cells with myeloma cells, which is called a hybridoma. These hybridomas have immortal grown properties of the myeloma cell and can secret antibodies due to the B-cells. As described above, an animal previously immunized against a CSP80, or an epitope thereof, has an enhanced population of B-lymphocytes that produce antibodies against the antigen. It is these cells that are fused with myeloma cells that do not produce antibodies themselves. This cell line is now cultured and is screened for the hybrids producing the desired antibodies. Screening may be performed by standard methods in the art, such as ELISA.

Monoclonal antibodies compatible to humans (humanized) can target human proteins such as CSP80 in vivo without adverse effects on patients. The same process is performed for the safe treatment of animals.

Antibodies to CSP80 may be used to measure these proteins in biological fluids and tissues using immunoassay that depends on the specificity of the monoclonal antibody. In the case of CSP80, the expression in cancers is unique and proves the involved cells or their fragments to be malignant.

Antibodies to CSP80 may be used to mark the proteins in specimens from test tissues. Conventional immunohistochemistry, known in the art, is carried out for this purpose. Mass spectroscopy and Western blotting are also used for detection in sample tissues.

ELISA, enzyme-linked immunosorbent assay, may be used for rapid qualitative diagnosis, in the format of conventional pregnancy tests. Providing an absorbent solid substrate, having a location to receive a biological sample potentially comprising CSP80 or a fragment thereof, the sample mobilizes on the absorbent solid substrate to interact and potentially bind to a mobile antibody, the conjugate further mobilizing to an immobile antibody, to form a CSP80 antigen sandwich, the immobile antibody providing a color change on the absorbent solid substrate in the presence of CSP80 or a fragment thereof. The monoclonal antibodies used in this assay may bind to CSP80 or any fragment thereof.

The instant invention provides a method to use the binding agents, such as monoclonal antibodies or small molecules, as detection agents for detecting and/or quantitating the CSP80 proteins in a number of pathological conditions, using samples such as body fluids, including but not limited to, peritoneal fluid, ascitic fluid, endometrial secretion, blood, serum, urine, semen, lymph fluid, and the link, which are obtained from an individual suffering from such proliferative conditions, or at risk of developing such conditions. Therefore, the sensitivity and specificity of the subject method are high, while false positive rate is low. The salient issue for the instant invention is that these conditions are marked by the presence of CSP80 while normal tissues are not. This use for finding abnormal tissues does not exclude the possibility that other conditions than mentioned will express CSP80 and therefore be identified. The instant invention is broadly applicable to conditions identifiable by the presence of CSP80, and are not only limited to the conditions listed by way of example in the instant application. As a result, the subject method can detect a low level of true positive signal, thus providing a method for early detection and diagnosis of diseases where early diagnosis is critical for prognosis.

The diagnostic method of the instant invention not only provides an early diagnosis/screening means for certain proliferative diseases, but also provide a non-invasive means to monitor the progress of the disease over time, its responsiveness to various treatments, and/or the possible recurrence of diseases previously in remission. Thus, the term “diagnosis” includes not only the initial diagnosis but also the monitoring of disease progression, the response of the disease to specific treatment regimens, the detection of possible recurrence, and screening of healthy individuals or individuals at high risk of developing the subject disease conditions, etc.

In the instant invention, CSP80 monoclonal antibodies are bound (affixed) to fluorescent or imaging-opaque molecules for the purpose of imaging to find their presence at the surgery or through the use of imaging. This is for the purpose of clinical identification, clinical staging for treatment or to evaluate the results of treatment. As described below, antibodies may also be bound to anti-cancer and proliferative drugs for the treatment of cancer or proliferative diseases. Monoclonal antibodies can be bound to nanoparticles and nanospheres by the monoclonal antibodies targeting the CSP80 in cancer or another proliferative disease. The nanoparticles may contain pharmaceutical treatments, radioactivity or other modalities for the treatment as well as the mapping of these conditions.

Small Molecules

The binding agent can be a small molecule antagonist of the CSP80 protein, such as those with molecular weights no more than about 5000 Da, 4000 Da, 3000 Da, 2000 Da, 1000 Da, 500 Da, 200 Da, or less than 100 Da. Such small molecule binding agents may be small peptides, or peptidio-mimetics, or any other organic or inorganic compounds that can bind any CSP80 epitope and inhibit the protein function (such as their role in proliferation and/or invasion, metastasis).

Computational modeling or other means may reveal extant and synthetic molecules (small molecules) that interact with CSP80 amino acid structures. The small molecules may bind to or interfere with the conformation or function of CSP80 or be attached to nanoparticles with cargos as described herein. In the case of direct action these small molecules are considered pharmaceutical agents in the instant application.

Monoclonal antibodies and small molecules against CSP80 may be attached to anti-cancer agents or nanoparticles to approximate them to pre-malignant, malignant, or proliferative disease cells. In such cases the therapeutic effects may be direct. Alternatively, the monoclonal antibody, small molecules or nanoparticles may be internalized, e.g. by endocytosis, after which they can target and attack abnormal protein expression or the cells that express the abnormal proteins.

### EXAMPLES

Various aspects of the instant invention are described below. The following examples are for illustrative purpose only and should in no way be construed as limiting in any respect of the claimed invention.

### Example 1

Conditioned Media (MS1303)

Sample Preparation: Each sample was concentrated to 2 mL or less by centrifuging each sample in a five kDa concentrator at 14,000 g for over two hours. Each serum sample was depleted using the Albumin Depletion kit (Pierce, catalog no. 85160) according to the manufacturer's protocol. The protein concentration was quantified by Qubit® fluorometry method (ThermoFisher, Rockville, Md.). 20 mpg of each was processed by SDS-PAGE using a 10% Bis-Tris NuPAGE mini-gel (Invitrogen) with the MES buffer system, the gel was run approximately 2 cm. The mobility region was excised into 20 equally sized bands and processed by in-gel digestion with trypsin using a ProGest robot Digilab, and washed with 25 mM ammonium bicarbonate followed by acetonitrile. Then reduced with 10 mM dithiothreitol at 60° C. followed by alkylation with 50 mM iodoacetamide at RT; and digested with trypsin (Promega) at 37° C. for 4 h, followed by quenching with formic acid. The supernatant was analyzed directly without further processing.

Mass Spectrometry: Half of each digested sample was analyzed by nano LC-MS/MS with a Waters M-Class HPLC system interfaced to a ThermoFisher Fusion Lumos mass spectrometer (ThermoFischer, Rockville, Md.). Peptides were loaded on a trapping column and eluted over a 75 μm analytical column at 350 nL/min; both columns were packed with Luna C18 resin (Phenomenex). The mass spectrometer was operated in data-dependent mode, with the Orbitrap operating at 60,000 FWHM and 15,000 FWHM for MS and MS/MS respectively. The instrument was run with a 3 s cycle for MS and MS/MS. 10 hrs of instrument time has used the analysis of each sample.

Data Processing: Data were searched using a local copy of Mascot (Matrix Science) with the following parameters:

### Enzyme: Trypsin/P;

Database: SwissProt Human (concatenated forward and reverse plus common contaminants);
Fixed modification: Carbamidomethyl (C);
Variable modifications: Oxidation (M), Acetyl (N-term), Pyro-Glu (N term Q), Deamidation (N/Q);
Mass values: Monoisotopic;
Peptide Mass Tolerance: 10 ppm;

### Fragment Mass Tolerance: 0.02 Da; and

### Max Missed Cleavages: 2.

Mascot DAT files were parsed into Scaffold (Proteome Software) for validation, filtering and to create a non-redundant list per sample. Data were filtered using 1% protein and peptide FDR and requiring at least two unique peptides per protein.

### Example 2

Serum Samples (MS1302)

Sample Preparation: Samples were pooled per client's instructions. Each serum sample was depleted using Proteome Purify 12 Human Serum Protein Immuno-Depletion Resin (R&D Systems, Catalog no. IDR012-020) according to the manufacturer's protocol. Depleted samples were buffer exchanged into water on a Corning Spin X 5 kD molecular weight cut off spin column and quantified by Qubit fluorometry (Life Technologies). 50 μg of each sample was reduced with dithiothreitol, alkylated with iodoacetamide and digested overnight with trypsin (Promega), and washed with 25 mM ammonium bicarbonate followed by acetonitrile. Then samples were reduced with 10 mM dithiothreitol at 60° C. followed by alkylation with 50 mM iodoacetamide at RT and digested with trypsin (Promega) at 37° C. for 4 h. The samples were quenched with formic acid and the supernatant was analyzed directly without further processing.

Mass Spectrometry: 2 ug of each sample was analyzed by nano LC-MS/MS with a Waters M-Class HPLC system interfaced to a ThermoFisher Fusion Lumos mass spectrometer. Peptides were loaded on a trapping column and eluted over a 75 μm analytical column at 350 nL/min; both columns were packed with Luna C18 resin (Phenomenex). A 4 hr gradient was employed. The mass spectrometer was operated in data-dependent mode, with the Orbitrap operating at 60,000 FWHM and 15,000 FWHM for MS and MS/MS respectively. The instrument was run with a 3 s cycle for MS and MS/MS.

Data Processing: Data were searched using a local copy of Mascot (Matrix Science) with the following parameters:
Enzyme: Trypsin/P
Database: SwissProt Human (concatenated forward and reverse plus common contaminants and appended with a custom sequence, see below)
Fixed modification: Carbamidomethyl (C)
Variable modifications: Oxidation (M), Acetyl (N-term), Pyro-Glu (N-term Q), Deamidation (N/Q)
Mass values: Monoisotopic
Peptide Mass Tolerance: 10 ppm
Fragment Mass Tolerance: 0.02 Da
Max Missed Cleavages: 2

### Example 3

Enzyme Linked ImmunoSorbant Assay (ELISA)

The assay may be done in various ways, including an Enzyme Linked ImmunoSorbent Assay (ELISA), in which a first immobilized binding agent (e.g., immobilized on a solid surface such as a 96-well plate, etc.) is used to bind and isolate CSP80 in a fluid sample, and a second detection binding agent (such as a binding agent labeled by a fluorescent dye, an enzyme, or a radio label) is used to bind the bound CSP80 protein. The presence and amount of the labeled second detection binding agent may then be determined/measured.

According to the subject method, the amount and/or concentration of the CSP80, or fragment thereof, detected in the sample is proportionally indicative of the severity and/or extent of the proliferative condition.

The diagnosis method of the invention may be performed, e.g., the amount and/or concentration of CSP80 is determined, using a binding agent which binds the CSP80. The binding agent may be an antibody, or a functional fragment thereof. “Functional” may only require the ability to bind in the context of the subject diagnosis methods. The antibody may be a polyclonal antibody or a monoclonal antibody. The antibody may be a xenogeneic antibody, an allogeneic antibody, or a syngeneic antibody. The antibody may be a modified antibody selected from the group consisting of: a chimeric antibody, a humanized antibody, and a fully human antibody. The functional fragment may be F(ab,Ä≤)2, Fab, Fv, scFv, or one or more CDR's.

In certain embodiments, the binding agent may also be tagged by a label, such as a fluorescent label, an enzyme label, or a radio-label.

The diagnosis methods of the invention may be used to detect CSP80 and fragments thereof.

In certain embodiments, the CSP80 binding agent may be labeled by a moiety, such as a fluorescent dye, an enzyme, or a radio-imaging reagent.

Sandwich Assay for ERM Protein Detection/Quantitation

A sandwich ELISA assay is used to detect and/or quantitate CSP80 in tissue sample/fluids. For example, to detect/quantitate CSP80 in a sample, binding agents such as a CSP80 capture antibody is bound to a 96-well plastic plate or absorbent solid substrate (or other solid support). CSP80 in samples is then captured and then detected/quantitated by a specific antibody. The third element in the “sandwich” is a species-specific anti-IgG that is labeled with an enzyme, such as peroxidase. The peroxidase reaction is developed and quantitated by an ELISA plate reader.

Therapeutic Compositions

Pharmaceutical or therapeutic compositions of the present invention are disclosed by way of example and not limitation. Those skilled in the art will be versed in making modifications of substitutions of various components, ingredients, dosages and treatment regimens.

The administration to a subject in need of the therapeutic pharmaceutical compositions of the present invention may be an intravenous infusion, oral ingestion, inhalation, intramuscular injection, subcutaneous injection, intravaginal application, and dermal and ocular penetration.

Pharmaceutical compositions for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an artemisinin-related compound as an active ingredient. An artemisinin-related compound may also be administered as a bolus, electuary or paste.

Nanoparticles in Therapeutic Compositions

Nanoparticles include endosomes, lipid rafts and low nanometer-sized artificial containers, known in the art, that carry cargos which may affect the targeted intracellular proteins or other vital intracellular molecules or structures. The cargo may be pharmaceuticals, natural interfering agents, synthetic molecules or radioactive materials. The agents may be auto fluorescent, x-ray or ultrasound opaque, etc. and be imaged directly at surgery or registered by imaging equipment.

The instant invention discloses targeting CSP80 by linking protein-specific monoclonal antibodies or binding small molecules to nanoparticles, or structures, small enough to bind to or enter living cells that express CSP80 proteins. The nanoparticles or other vesicles, including lipid rafts, that carry cargos such as radioactive agents, chemotherapeutic drugs, or other agents that sabotage cell function, thereby inactivating or destroying these cells. In this case the antibodies, nanoparticles or small molecules act as drug delivery systems. In addition, once they have been linked to the CSP80 it is not necessary that CSP80 be affected by the monoclonal antibodies, nanoparticles or small molecules, themselves. Rather, the attached or cargo agents may be the treatment. The small molecules, by binding to the target proteins may themselves inactivate them. The affected cells are then disposed of by the usual cellular mechanisms including apoptosis, autophagy, etc.

The complexed nanoparticles may be of a size ranging from 0.1 μm and 1.0 μm. The shape of the nanoparticle may be a sphere, cuboidal or elongated depending on the desired permeability characteristics of a target cell. Nanoparticles also have surface functionality. For instance, to bind poly-ethylene-glycol to increase circulation time to prolong the therapeutic effect of the complexed cargo on the nanoparticle.

The ability to deliver multiple copies of a radiotherapeutic to a single receptor site is perhaps the most useful property that can be combined using a nanoconjugate, which consists of a nanoparticle, a linking agent, and an antibody or peptide. Other useful properties include tuning the biodistribution by altering the nanoparticle's surface. When compared to currently approved targeted radiotherapies, the cytotoxicity of nanoparticle-based medicines will be higher when many radioactive atoms are delivered to each receptor. Modular surface modification enables the nanoparticle system to be biodistributed specifically to enhance accumulation at the tumor site.

Antibody labeling of gold-coated lanthanide phosphate nanoparticles can produce promising theragnostic anti-cancer nanoconjugates. The intermediate energy beta emitted in the decay can be quite effective in treating metastatic disease. The 208 keV gamma-ray from 177Lu decay (11%) can be employed for SPECT imaging of the radiotherapeutic drug. Each nanoparticle would contain three radioactive atoms on average if 20 mCi of activity were used in the synthesis.

Protocols for conjugating CSP80 binding agents to nanoparticles, or therapeutic agents to nanoparticles, will vary based on the selected nanoparticles. Commercial kits are also available to facilitate this complex.

By way of example and not limitation, the cargo on the nanoparticle may be a therapeutic pharmaceutical agent selected from the group consisting of: methotrexate, amsacrine, azacytidine, bleomycin, busulfan, capecitabine, carboplatin, carmustine, chlorambucil, cisplatin, cladribine, cyclophosphamide, cytarabine, dactinomycin, daunombicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, vinorelbine, aminoglutethimide, anastrozole, asparaginase, bcg, bicalutamide, buserelin, campothecin, clodronate, colchicine, cyproterone, dacarbazine, dienestrol, diethylstilbestrol, estradiol, exemestane, filgrastim, fludrocortisone, fluoxymesterone, flutamide, genistein, goserelin, hydroxyurea, imatinib, interferon, ironotecan, letrozole, leucovorin, leuprolide, levamisole, medroxyprogesterone, megestrol, mesna, nilutamide, nocodazole, octreotide, pamidronate, porfimer, raltitrexed, rituximab, suramin, tamoxifen, temozolomide, testosterone, titanocene dichloride, trastuzumab, tretinoin, vindesine, HERCEPTIN® and other antibody therapeutics, and an anti-sense or RNAi agent against one or more genes promoting the progression of the cancer.

Multiple therapeutic agents and multiple CSP80-binding agents may be complexed to a nanoparticle, determined by the condition being treated the severity, aggressiveness of the progression of the proliferative condition and other clinical variables. It will be obvious to those skilled in the art to vary the complexed components to increase specificity and therapeutic value of a nanoparticle treatment.

### EQUIVALENTS

Those skilled in the art will recognize or be able to ascertain using no more than routine experimentation, numerous equivalents to the specific method and reagents described herein, including alternatives, variants, additions, deletions, modifications, and substitutions. Cancer or proliferative diseases in non-human species expressing CSP80 are targets for the same agents as described above.

Subjects of the invention may be any human, including pregnant women. The instant invention is also applicable to domesticated animals, or non-domesticated animals. The uses of the present invention may be used in human medicine and veterinary medicine.

1. A method of detecting a proliferative condition in a subject, comprising:
a) obtaining a biological sample from an animal subject;
b) subjecting said biological sample to an anti-CSP80 binding agent; and
c) detecting a binding interaction between CSP80 and an anti-CSP80 binding agent;
wherein the binding of CSP80 and anti-CSP80 binding agent indicates the presence of CSP80.

2. The method of claim 1, further comprising detecting said binding interaction between CSP80 and anti-CSP80 binding agent with an ELISA assay.

3. The method of claim 1, further comprising detecting said binding interaction between CSP80 and anti-CSP80 binding agent with mass spectrometry.

4. The method of claim 1, further comprising detecting said binding interaction between CSP80 and anti-CSP80 binding agent with a western blot assay.

5. The method of claim 1, wherein the presence of CSP80 indicates that said animal subject has a proliferative condition.

6. The method of claim 5, wherein said proliferative condition is selected from the group consisting of cancer, metastatic cancer, non-metastatic cancer, ovarian epithelial cancer (OVCA), Endometrial adenocarcinoma (ENDOCA), or Cervical cancer (CXCA), ovarian cancer, endometrial cancer, breast cancer, glioblastoma, schwannoma, meningioma, malignant mesothelioma, neurofibromatosis, colon cancer, oral cancer, or cancer selected from the group consisting of: lung cancer, prostate cancer, pancreatic cancer, leukemia, liver cancer, stomach cancer, uterine cancer, testicular cancer, brain cancer, non-Hodgkin's lymphoma, Hodgkin's lymphoma, Ewing's sarcoma, osteosarcoma, neuroblastoma, rhabdomyosarcoma, melanoma, and brain cancer, blood cancers, fibromatoses, psoriasis, meningiomas rheumatoid arthritis, atherosclerosis, idiopathic pulmonary fibrosis, scleroderma, and cirrhosis of the liver and placentation/gestation.

7. The method of claim 1, wherein the binding agent is selected from the group consisting of a small molecule, a monoclonal antibody, a polyclonal antibody, or a functional fragment thereof.

8. The method of claim 1, wherein said animal subject is selected from the group consisting of a human, a non-human animal, a pregnant human and a pregnant non-human animal.

9. The method of claim 1, wherein said biological sample is selected from a group consisting of bodily fluid or contents thereof, ascitic fluid, endometrial secretion, blood, urine, serum, lymph fluid, endometrial washing, semen, solid tissue and or contents thereof, and combinations thereof.

10. The method of claim 1, wherein the detection of said binding interaction is to assess the efficacy of a treatment.

11. The method of claim 1, wherein the detection of said binding interaction is to assess the progress of placentation and intrauterine growth.

12. The method of claim 1, wherein the detection of said binding interaction is to assess the progress of proliferation of during gestation and organ function.

13. The method of claim 1, further comprising a label being complexed to said CSP80 binding agent.

14. The method of claim 13, wherein said label is selected from the group consisting of a fluorescent label, radioactive label, chemiluminescent label and combinations thereof.

15. The method of claim 13, wherein said CSP80 binding agent complexed to said label is used in imaging said animal subject for screening of a proliferative condition.
